(19) United States
(12) Patent Application Publication
(10) Pub. No.: US 2013/0005764 A1
(43) Pub. Date: Jan. 3, 2013

(54) QUININE DOSAGE FORMS AND METHODS OF USE THEREOF

(76) Inventor: Jie Du, Lansdale, PA (US)

(21) Appl. No.: 13/610,938

(22) Filed: Sep. 12, 2012

Related U.S. Application Data
(63) Continuation of application No. 12/468,246, filed on May 19, 2009.
Application No. 12/468,246 is a continuation of application No. 11/415,847, filed on May 2, 2006, now abandoned.

(51) Int. Cl. A61K 31/49; A61P 21/00; A61P 33/02; A61P 33/06

(52) U.S. Cl. 514/305

(57) ABSTRACT

Disclosed herein are quinine formulations and methods of using quinine formulations. Specifically disclosed are methods of using quinine and informing a user of information, including potential adverse effects, the effect of food on quinine's pharmacokinetics, effect of dosing various strengths of quinine, effect of maximum plasma concentrations of quinine in a patient as it relates to adverse events, effects of deviating from the prescribed dosage, etc.

### CROSS-REFERENCE TO RELATED APPLICATIONS

This application is a continuation of U.S. patent application Ser. No. 12/468,246, filed May 19, 2009, which is a continuation of U.S. patent application Ser. No. 11/415,847, filed May 2, 2006, the disclosures of both are incorporated herein by reference in their entirety; and this application claims the benefit of U.S. Provisional Application Ser. No. 60/729,574, filed Oct. 24, 2005 and U.S. Provisional Application Ser. No. 60/677,269, filed May 3, 2005, both of which are incorporated by reference in their entirety.

### BACKGROUND

Malaria is a parasitic disease caused by the Plasmodium species P. falciparum, P. vivax, P. ovale and P. malariae. The malaria parasite causes intermittent fevers and chills. It affects multiple organs and systems, including red blood cells, the kidneys, liver, spleen, and brain. It is estimated by the World Health Organization (WHO) that up to 500 million persons per year are infected with malaria, with 200 to 300 million people suffering from malaria at any given time (See Roll Back Malaria, World Health Organization, available at: www.rbm.who.int/cmc_upload/0/000/015372/RBMInfosheet_{—}1.htm). Up to 3 million will die each year. If P. falciparum infection goes untreated or is not treated appropriately, general observations indicate that mortality is high, killing up to 25% of non-immune adults within 2 weeks of a primary attack [Taylor T E, Strickland G T. Malaria. In: Strickland GT, ed. Hunter's Tropical Medicine and Emerging Infectious Diseases. 8th ed. Philadelphia, Pa.: W.B. Saunders Company; 2000.] A significant number of these cases are found in Central America, South America, Asia, and Africa. Known antimalarial agents include 9-aminoacridines (e.g. mepacrine), 4-aminoquinolines (e.g. amodiaquine, chloroquine, hydroxychloroquine), 8-aminoquinolines (e.g. primaquine, quinocide), biguanides with an inhibiting effect on dihydrofolic acid reductase (e.g. chlorproguanil, cycloguanil, proguanil), diaminopyrimidines (e.g. pyrimethamine), quinine salts, sulphones such as dapsone, sulphonamides, sulphanilamides and antibiotics such as tetracycline.

Quinine (cinchonan-9-ol, 6′-methoxy-, (8α,9R)-) is an antiprotozoal and an antimyotonic, and is known for the treatment of malaria caused by Plasmodium species, the treatment and prophylaxis of nocturnal recumbency leg muscle cramps, and the treatment of babesiosis caused by Babesia microti. Quinine is structurally similar to quinidine, which is also an antiprotozoal, but can function as an antiarrhythmic. Quinidine has been associated with the prolongation of the QT interval in a dose-related fashion. Prolongation of the electrocardiographic QT interval can be indicative of delayed ventricular repolarization. Excessive QT prolongation has been associated with an increased risk of ventricular arrhythmia. Although quinine is a diastereomer of quinidine, it does not cause QT prolongation to the same degree although it has been suggested that patients with a history of cardiac arrhythmias or QT prolongation should carefully consider taking quinine as they may be at risk for arrhythmias.

There remains a need in the art for methods of dosing quinine formulations that provide a desired therapeutic effect against certain diseases (e.g., malaria) while at the same time minimizing the potential adverse side effects associated with dosing of quinine such as QT prolongation. There accordingly remains a need in the art for improved methods for the administration of quinine.

### SUMMARY

Disclosed herein are methods of using quinine, methods of manufacturing a quinine pharmaceutical product and an article prepared therefrom. Also disclosed herein are methods of preventing or treating malaria; preventing or treating leg cramps, including for example nocturnal recumbency leg muscle cramps, idiopathic leg cramps, and leg cramps caused by athletic exertion; or babesiosis caused by Babesia microti.

In one embodiment, a method of using quinine comprises informing a user: a) that administration of quinine provides a mean±SD maximum QTc change from baseline around the quinine Tₘₐₓ of 10±19 msec for a 324 mg dose of quinine sulfate and 12±18 msec for a 648 mg dose of quinine sulfate; b) that a maximum QTc change from baseline of no greater than about 60 msec is observed in patients receiving a 324 mg dose of quinine sulfate or a 648 mg dose of quinine sulfate; c) that a QTc interval of no greater than about 500 msec is experienced in patients receiving a 324 mg dose of quinine sulfate or a 648 mg dose of quinine sulfate; or d) that the maximum increase in QTc interval corresponds with peak quinine plasma concentration (Cₘₐₓ).

In another embodiment, a method of using quinine comprises obtaining quinine from a container providing information a) that administration of quinine provides a mean±SD maximum QTc change from baseline around the quinine Tₘₐₓ of 10±19 msec for a 324 mg dose of quinine sulfate and 12±18 msec for a 648 mg dose of quinine sulfate; b) that a maximum QTc change from baseline of no greater than about 60 msec is observed in patients receiving a 324 mg dose of quinine sulfate or a 648 mg dose of quinine sulfate; c) that a QTc interval of no greater than about 500 msec is experienced in patients receiving a 324 mg dose of quinine sulfate or a 648 mg dose of quinine sulfate; or d) that the maximum increase in QTc interval corresponds with peak quinine plasma concentration (Cₘₐₓ).

In yet another embodiment, a method of manufacturing a quinine pharmaceutical product comprises packaging a quinine dosage form with information a) that administration of quinine provides a mean±SD maximum QTc change from baseline around the quinine Tₘₐₓ of 10±19 msec for a 324 mg dose of quinine sulfate and 12±18 msec for a 648 mg dose of quinine sulfate; b) that a maximum QTc change from baseline of no greater than about 60 msec is observed in patients receiving a 324 mg dose of quinine sulfate or a 648 mg dose of quinine sulfate; c) that a QTc interval of no greater than about 500 msec is experienced in patients receiving a 324 mg dose of quinine sulfate or a 648 mg dose of quinine sulfate; or d) that the maximum increase in QTc interval corresponds with peak quinine plasma concentration (Cₘₐₓ).

In still yet another embodiment, a method of using quinine comprises providing a user with quinine; and informing the user a) that administration of quinine provides a mean±SD maximum QTc change from baseline around the quinine Tₘₐₓ of 10±19 msec for a 324 mg dose of quinine sulfate and 12±18 msec for a 648 mg dose of quinine sulfate; b) that a maximum QTc change from baseline of no greater than about 60 msec is observed in patients receiving a 324 mg dose of quinine sulfate or a 648 mg dose of quinine sulfate; c) that a QTc interval of no greater than about 500 msec is experienced in patients receiving a 324 mg dose of quinine sulfate or a 648 mg dose of quinine sulfate; or d) that the maximum increase in QTc interval corresponds with peak quinine plasma concentration (Cₘₐₓ).

In one embodiment, an article of manufacture comprises a container containing a dosage form of quinine, wherein the container is associated with published material informing a) that administration of quinine provides a mean±SD maximum QTc change from baseline around the quinine Tₘₐₓ of 10±19 msec for a 324 mg dose of quinine sulfate and 12±18 msec for a 648 mg dose of quinine sulfate; b) that a maximum QTc change from baseline of no greater than about 60 msec is observed in patients receiving a 324 mg dose of quinine sulfate or a 648 mg dose of quinine sulfate; c) that a QTc interval of no greater than about 500 msec is experienced in patients receiving a 324 mg dose of quinine sulfate or a 648 mg dose of quinine sulfate; or d) that the maximum increase in QTc interval corresponds with peak quinine plasma concentration (Cₘₐₓ).

In another embodiment, a method of using quinine comprises administering a quinine formulation to a patient; wherein the administering provides a therapeutic plasma concentration of quinine, and wherein a) the patient experiences a QTc interval of no greater than about 550 msec; or b) the patient experiences a maximum QTc change from baseline of no greater than about 60 msec.

In yet another embodiment, a method of using quinine comprises informing a user: a) that the oral administration of quinine under fed conditions does not substantially affect Cₘₐₓ or AUC of quinine when compared to fasted conditions; b) that the oral administration of about 324 or about 648 mg of quinine sulfate under fed conditions does not substantially affect Cₘₐₓ or AUC of quinine when compared to fasted conditions; c) that the oral administration of quinine under fed conditions increases Tₘₐₓ of quinine when compared to fasted conditions; or d) that the oral administration of about 324 or about 648 mg of quinine sulfate under fed conditions increases Tₘₐₓ of quinine when compared to fasted conditions.

In still yet another embodiment, a method of using quinine comprises obtaining quinine from a container providing information a) that the oral administration of quinine under fed conditions does not substantially affect Cₘₐₓ or AUC of quinine when compared to fasted conditions; b) that the oral administration of about 324 or about 648 mg of quinine sulfate under fed conditions does not substantially affect Cₘₐₓ or AUC of quinine when compared to fasted conditions; c) that the oral administration of quinine under fed conditions increases Tₘₐₓ of quinine when compared to fasted conditions; or d) that the oral administration of about 324 or about 648 mg of quinine sulfate under fed conditions increases Tₘₐₓ of quinine when compared to fasted conditions.

In one embodiment, a method of manufacturing a quinine pharmaceutical product comprises packaging a quinine dosage form with information a) that the oral administration of quinine under fed conditions does not substantially affect Cₘₐₓ or AUC of quinine when compared to fasted conditions; b) that the oral administration of about 324 or about 648 mg of quinine sulfate under fed conditions does not substantially affect Cₘₐₓ or AUC of quinine when compared to fasted conditions; c) that the oral administration of quinine under fed conditions increases Tₘₐₓ of quinine when compared to fasted conditions; or d) that the oral administration of about 324 or about 648 mg of quinine sulfate under fed conditions increases Tₘₐₓ of quinine when compared to fasted conditions.

In another embodiment, a method of using quinine comprises providing a user with quinine; and informing the user a) that the oral administration of quinine under fed conditions does not substantially affect Cₘₐₓ or AUC of quinine when compared to fasted conditions; b) that the oral administration of about 324 or about 648 mg of quinine sulfate under fed conditions does not substantially affect Cₘₐₓ or AUC of quinine when compared to fasted conditions; c) that the oral administration of quinine under fed conditions increases Tₘₐₓ of quinine when compared to fasted conditions; or d) that the oral administration of about 324 or about 648 mg of quinine sulfate under fed conditions increases Tₘₐₓ of quinine when compared to fasted conditions.

In one embodiment, an article of manufacture comprises a container containing a dosage form of quinine, wherein the container is associated with published material informing a) that the oral administration of quinine under fed conditions does not substantially affect Cₘₐₓ or AUC of quinine when compared to fasted conditions; b) that the oral administration of about 324 or about 648 mg of quinine sulfate under fed conditions does not substantially affect Cₘₐₓ or AUC of quinine when compared to fasted conditions; c) that the oral administration of quinine under fed conditions increases Tₘₐₓ of quinine when compared to fasted conditions; or d) that the oral administration of about 324 or about 648 mg of quinine sulfate under fed conditions increases Tₘₐₓ of quinine when compared to fasted conditions.

In an embodiment, a method of using quinine comprises administering a quinine formulation with a high fat meal, wherein the time to achieve Tₘₐₓ is about 15 minutes to about 4.0 hours greater than a Tₘₐₓ achieved under fasted conditions.

In an embodiment, a method of using quinine comprises informing a user a) that the plasma levels of quinine sulfate when dosed orally under fasted conditions displays non-linear pharmacokinetics (AUC and Cₘₐₓ) when dosed between about 324 mg and about 648 mg; or b) that the plasma levels of the quinine sulfate when dosed orally under fasted conditions displays substantially linear pharmacokinetics (AUC and Cₘₐₓ) when dosed between about 260 mg to about 324 mg.

In an embodiment, a method of using quinine comprises obtaining quinine from a container providing information a) that the plasma levels of quinine sulfate when dosed orally under fasted conditions displays non-linear pharmacokinetics (AUC and Cₘₐₓ) when dosed between about 324 mg and about 648 mg; or b) that the plasma levels of the quinine sulfate when dosed orally under fasted conditions displays substantially linear pharmacokinetics (AUC and Cₘₐₓ) when dosed between about 260 mg to about 324 mg.

In one embodiment, a method of manufacturing a quinine pharmaceutical product comprises packaging a quinine dosage form with information a) that the plasma levels of quinine sulfate when dosed orally under fasted conditions displays non-linear pharmacokinetics (AUC and Cₘₐₓ) when dosed between about 324 mg and about 648 mg; or b) that the plasma levels of the quinine sulfate when dosed orally under fasted conditions displays substantially linear pharmacokinetics (AUC and Cₘₐₓ) when dosed between about 260 mg to about 324 mg.

In an embodiment, a method of using quinine comprises providing a user with quinine; and informing the user a) that the plasma levels of quinine sulfate when dosed orally under fasted conditions displays non-linear pharmacokinetics (AUC and Cₘₐₓ) when dosed between about 324 mg and about 648 mg; or b) that the plasma levels of the quinine sulfate when dosed orally under fasted conditions displays substantially linear pharmacokinetics (AUC and Cₘₐₓ) when dosed between about 260 mg to about 324 mg.

In one embodiment, an article of manufacture comprising a container containing a dosage form of quinine, wherein the container is associated with published material informing a) that the plasma levels of quinine sulfate when dosed orally under fasted conditions displays non-linear pharmacokinetics (AUC and Cₘₐₓ) when dosed between about 324 mg and about 648 mg; or b) that the plasma levels of the quinine sulfate when dosed orally under fasted conditions displays substantially linear pharmacokinetics (AUC and Cₘₐₓ) when dosed between about 260 mg to about 324 mg.

### BRIEF DESCRIPTION OF THE DRAWINGS

FIG. 1 Mean plasma concentrations and QTc measurements over 24-hours following a single oral dose of Quinine Sulfate under fasting conditions;

FIG. 2 Mean plasma concentrations and QTc measurements over 24-hours following a single oral dose of Quinine Sulfate under fed conditions;

FIG. 3 Mean plasma concentration and QTc measurements over 24-hours following a single oral dose of Quinine Sulfate 324 mg under fasting conditions;

FIG. 4 Mean plasma concentration and QTc measurements over 24-hours following a single oral dose of Quinine Sulfate 648 mg under fasting conditions.

### DETAILED DESCRIPTION

Disclosed herein are quinine formulations, kits, and methods of using quinine and quinine formulations. Specifically disclosed are methods of using quinine and informing the user of certain information. Such information can include, for example, the effect of food on quinine's pharmacokinetics, effect of dosing various strengths of quinine, effect of maximum plasma concentrations of quinine in a patient as it relates to adverse effects including QT prolongation, effects of deviating from the prescribed dosage, etc. With the knowledge of the particular information, the administration of quinine to the patient can be optimized to provide safer use of quinine, while oftentimes reducing or minimizing side effects or adverse events.

Quinine therapy can be considered optimal when effective plasma levels are reached when required. In addition, peak plasma values (Cₘₐₓ) should be as low as possible so as to reduce the incidence and severity of possible side effects, including the adverse event of QT prolongation. QT prolongation events can be monitored by surface electrocardiogram (EKG) measured from the beginning of the QRS complex to the end of the T wave, which represents the duration of activation and recovery of the ventricular myocardium. The QT values can be heart rate corrected to “QTc”. Generally, a QTc above about 0.44 seconds is considered abnormal, although there are age- and sex-specific abnormal QTc values which vary from this number.

As used herein, the term “wherein administration of a quinine or a quinine formulation does not cause significant QT prolongation according to the standards of the United States Food and Drug Administration” means the standards found in the document Guidance for Industry, E14 Clinical Evaluation of QT/QTc Interval Prolongation and Proarrhythmic Potential for Non-Antiarrhythmic Drugs, U.S. Department of Health and Human Services Food and Drug Administration, Center for Drug Evaluation and Research (CDER), Center for Biologics Evaluation and Research (CBER) issued Oct. 2005 and available at http://www.fda.gov/cder/guidance/index.htm.

The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”). The endpoints of all ranges directed to the same component or property are inclusive and independently combinable.

An “active agent” means a compound, element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates) of the free compound or salt, crystalline forms, non-crystalline forms, and any polymorphs of the compound are contemplated herein.

“Pharmaceutically acceptable salts” include derivatives of the active agent (e.g. quinine), wherein the parent compound is modified by making non-toxic acid addition salts thereof, and further refers to pharmaceutically acceptable solvates, including hydrates, of such compounds and such salts. Also included are all crystalline, amorphous, and polymorph forms. Examples of pharmaceutically acceptable salts include, but are not limited to, mineral or organic acid addition salts; and the like, and combinations comprising one or more of the foregoing salts. The pharmaceutically acceptable salts include non-toxic salts, for example, from non-toxic inorganic or organic acids. For example, non-toxic acid salts include those derived from inorganic acids such as hydrochloric, hydrobromic, sulfuric, sulfamic, phosphoric, nitric and the like. Pharmaceutically acceptable organic salts includes salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Specific quinine salts include quinine sulfate, quinine hydrochloride, quinine dihydrochloride, and hydrates or solvates thereof.

“Quinine” as used herein is inclusive of all pharmaceutically acceptable salt forms, crystalline forms, amorphous form, polymorphic forms, solvates, and hydrates unless specifically indicated otherwise. As used herein, “quinine sulfate” means cinchonan-9-ol, 6′-methoxy-, (8α,9R)-, sulfate (2:1) or cinchonan-9-ol, 6′-methoxy-, (8α,9R)-, sulfate (2:1) dehydrate unless otherwise indicated.

“Bioavailability” means the extent or rate at which an active agent is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” can be characterized by one or more pharmacokinetic parameters.

A “dosage form” means a unit of administration of an active agent. Examples of dosage forms include tablets, capsules, injections, suspensions, liquids, emulsions, creams, ointments, suppositories, inhalable forms, transdermal forms, and the like. The quinine formulation may be a dosage form administered via oral, buccal, injectable, or transdermal administration. The dosage form is not particularly limited and can be any one of the following forms: immediate-release, controlled-release, extended-release, sustained-release, pulsed-release, delayed-release, and the like.

By an “effective” amount or a “therapeutically effective amount” of an active agent is meant a sufficient amount of the active agent to produce a therapeutic effect in the patient. The amount that is “effective” will vary from subject to subject, depending on the age and general condition of the individual, the particular active agent, and the like. Thus, it is not always possible to specify an exact “effective amount.” However, an appropriate “effective” amount in any individual case may be determined by one of ordinary skill in the art using routine experimentation.

“Efficacy” means the ability of an active agent administered to a patient to produce a therapeutic effect in the patient.

“Enhancing the safety profile” of an active agent means implementing actions or articles designed or intended to help reduce the incidence of adverse events associated with administration of the active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

“Informing” means referring to or providing, published material, for example, providing an active agent with published material to a user; or presenting information orally, for example, by presentation at a seminar, conference, or other educational presentation, by conversation between a pharmaceutical sales representative and a medical care worker, or by conversation between a medical care worker and a patient; or demonstrating the intended information to a user for the purpose of comprehension.

A “medical care worker” means a worker in the health care field who may need or utilize information regarding an active agent including a dosage form thereof, including information on safety, efficacy, dosing, administration, or pharmacokinetics. Examples of medical workers include physicians, pharmacists, physician's assistants, nurses, aides, caretakers (which can include family members or guardians), emergency medical workers, and veterinarians.

A “patient” means a human or non-human animal in need of medical treatment. Medical treatment can include treatment of an existing condition, such as a disease or disorder, prophylactic or preventative treatment, or diagnostic treatment. In some embodiments the patient is a human patient. The terms “treating” and “treatment” mean implementation of therapy with the intention of reducing in severity or frequency symptoms, elimination of symptoms or underlying cause, prevention of the occurrence of symptoms or their underlying cause, and improvement or remediation of damage.

A “pharmaceutical supplier” means a person (other than a medical care worker), business, charitable organization, governmental organization, or other entity involved in the transfer of active agent, including a dosage form thereof, between entities, for profit or not. Examples of pharmaceutical suppliers include pharmaceutical distributors, pharmacy chains, pharmacies (online or physical), hospitals, HMOs, supermarkets, the Veterans Administration, or foreign businesses or individuals importing active agent into the United States.

“Pharmacokinetic parameters” describe the in vivo characteristics of an active agent (or surrogate marker for the active agent) over time, such as plasma concentration (C), Cₘₐₓ, Cₙ, C₂₄, Tₘₐₓ, and AUC. “Cₘₐₓ” is the measured concentration of the active agent in the plasma at the point of maximum concentration. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) vs. time, measured from one time point to another time point. For example AUC₀₋ₜ is the area under the curve of plasma concentration versus time from time 0 to time t. The AUC_{0-∞} or AUC_{0-INF} is the calculated area under the curve of plasma concentration versus time from time 0 to time infinity.

A “product” or “pharmaceutical product” means a dosage form of an active agent plus published material and optionally packaging.

“Providing” means giving, administering, selling, distributing, transferring (for profit or not), manufacturing, compounding, or dispensing.

“Published material” means a medium providing information, including printed, audio, visual, or electronic medium, for example a flyer, an advertisement, a product insert, printed labeling, an internet web site, an internet web page, an internet pop-up window, a radio or television broadcast, a compact disk, a DVD, an audio recording, or other recording or electronic medium.

“Safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

A “user” means a patient, a medical care worker, or a pharmaceutical supplier.

Disclosed herein are methods of using quinine, which may provide an increase in the safety or efficacy of the quinine treatment. Extensive research has been performed with administering quinine that now reveal several developments relating to the improvement in the safe and effective treatment using quinine.

QTc interval prolongation was evaluated in a crossover pharmacokinetic study in healthy volunteers (n=24) who received single oral doses of quinine sulfate (324 mg and 648 mg). The mean±SD maximum QTc change from baseline around the quinine Tmax were found to be 10±19 msec and 12±18 msec, respectively for the 324 mg and 648 mg doses. There were no subjects who had a QTc interval greater than 500 msec, or with a maximum QTc change from baseline of greater than 60 msec. The study reveals that the maximum increase in QTc interval has been shown to correspond with peak quinine plasma concentration. Accordingly, it is recommended that quinine sulfate be used with caution when taken by a patient who is also taking other substances known to cause QT prolongation. These other substances include, for example, Class IA antiarrhythmic agents (e.g. quinidine, procainamide, disopyramide), and Class III antiarrhythmic agents (e.g. amiodarone, sotalol, dofetilide).

Based on the results of the study, especially that the mean maximum increase in QTc interval corresponds with mean peak quinine plasma concentration (Cₘₐₓ), by informing a user of the studies' results can help to provide an increase in the safe administration of quinine and quinine formulations. In one embodiment, use of quinine comprises providing a quinine dosing regimen in such a way as to result in an appropriate therapeutic plasma concentration of quinine in the patient, while at the same time controlling the dosing such that the patient will experience a QTc interval of no greater than about 550 msec, specifically no greater than about 500 msec, more specifically no greater than about 450 msec, and yet more specifically no greater than about 425 msec. Furthermore, in addition to the administration of the quinine formulation, the user is informed that the maximum increase in QTc interval corresponds with peak quinine plasma concentration (Cₘₐₓ). Such dosing regimens can be provided by one of ordinary skill in the art, taking into consideration such factors as the age, sex, and health of the patient, as well as medications the patient may be taking at the time. Optionally, the patient can be monitored at the initial stages of treatment to ensure therapeutic plasma levels of quinine that do not result in QTc intervals greater than described herein.

In another embodiment, the use of quinine comprises providing a quinine dosing regimen in such a manner as to result in an appropriate therapeutic plasma concentration of quinine in the patient, while at the same time the patient will experience a maximum QTc change from baseline of less than about 60 msec, specifically less than about 40 msec, and yet more specifically less than about 20. Additionally, the user is informed that the maximum increase in QTc interval corresponds with peak quinine plasma concentration (Cₘₐₓ).

As it has been found that the QT interval corresponds to peak quinine plasma concentration, use of quinine includes informing the user of certain procedures to limit or reduce the possibility of a spike in quinine plasma concentration or elevated plasma levels in excess of therapeutic plasma levels. By minimizing or reducing the likelihood of these spikes or elevations in quinine plasma concentration, the possibility of an increase in QT prolongation may also minimized or reduced. Such nonlimiting procedures include, for example, informing the user that the patient should not take more than a prescribed dose of quinine; wherein a dose should not be doubled when a dose is missed; when administered quinine, the patient should not take more than 538 mg free base equivalent (648 mg quinine sulfate) at one time, or more than three doses in one day (538 mg free base equivalent TID or 648 mg quinine sulfate TID), or more than 1614 mg free base equivalent (1944 mg quinine sulfate) in one day; that if the patient has missed a dose and more than about four hours has elapsed since the missed dose, it is recommended that the missed dose not be taken, but the patient is recommended take the next dose as previously scheduled; that caution is recommended when quinine is administered with a substance known to cause QT prolongation or known to otherwise affect the pharmacokinetics of quinine, such as a Class IA antiarrhythmic agents (e.g. quinidine, procainamide, disopyramide) or Class III antiarrhythmic agents (e.g. amiodarone, sotalol, dofetilide), astemizole, cisapride, terfenadine, pimozide, halofantrine, quinidine, mefloquine, or halofantrine; informing the patient's physician of any prescription or non-prescription medication that the patient is taking; that caution is recommended if quinine is administered to a patient known to have prolongation of QTc interval; that caution is recommended if quinine is administered to an elderly patient; that caution is recommended if quinine is administered to a patient with a clinical condition known to prolong the QT interval, such a conditions includes uncorrected hypokalemia, bradycardia, or cardiac conditions; that caution is recommended if quinine is administered with a substance that inhibits the metabolism of quinine; or that it is recommended that a patient be monitored for adverse reactions when the patient is administered quinine concomitantly with a substance that inhibits the metabolism of quinine.

In one embodiment, a method of using quinine comprises informing a user a) that administration of quinine provides a mean±SD maximum QTc change from baseline around the quinine Tₘₐₓ of 10±19 msec for a 324 mg dose of quinine sulfate and 12±18 msec for a 648 mg dose of quinine sulfate; b) that a maximum QTc change from baseline of no greater than about 60 msec is observed in patients receiving a 324 mg dose of quinine sulfate or a 648 mg dose of quinine sulfate; c) that a QTc interval of no greater than about 500 msec is experienced in patients receiving a 324 mg dose of quinine sulfate or a 648 mg dose of quinine sulfate; or d) that the maximum increase in QTc interval corresponds with peak quinine plasma concentration (Cₘₐₓ).

In another embodiment, a method of using quinine comprises obtaining quinine from a container providing information a) that administration of quinine provides a mean±SD maximum QTc change from baseline around the quinine Tₘₐₓ of 10±19 msec for a 324 mg dose of quinine sulfate and 12±18 msec for a 648 mg dose of quinine sulfate; b) that a maximum QTc change from baseline of no greater than about 60 msec is observed in patients receiving a 324 mg dose of quinine sulfate or a 648 mg dose of quinine sulfate; c) that a QTc interval of no greater than about 500 msec is experienced in patients receiving a 324 mg dose of quinine sulfate or a 648 mg dose of quinine sulfate; or d) that the maximum increase in QTc interval corresponds with peak quinine plasma concentration (Cₘₐₓ).

In yet another embodiment, a method of using quinine comprises administering a quinine formulation to a patient; wherein the administering provides a therapeutic plasma concentration of quinine, and wherein a) the patient experiences a QTc interval of no greater than about 550 msec; or b) the patient experiences a maximum QTc change from baseline of no greater than about 60 msec.

Further studies have recently found that oral administration of a quinine sulfate formulation results in the extension of Tₘₐₓ under fed conditions as compared to fasted conditions (percent ratio of fed:fasted was found to be 153.25%, n=24), however, the Cₘₐₓ and AUC₀₋ₜ remained substantially unchanged between the fed and fasted states. In one embodiment, use of quinine comprises informing the user that quinine can be taken with or without food. To improve patient compliance, however, the user can be informed that quinine can be taken with or without food, but to reduce the incidence of gastric upset, it is recommended that the patient take quinine with food.

In another embodiment, quinine can be administered orally to a patient with a meal, specifically a high fat meal, to result in the further extension of Tₘₐₓ when compared to the Tₘₐₓ achieved in the fasted state. The Tₘₐₓ achieved by quinine administered with a meal can be about 3 hours or greater, specifically about 4 hours or greater, and more specifically about 5 hours or greater. An exemplary high fat meal includes the test meal disclosed in the document Guidance for Industry, Food-Effect Bioavailability and Fed Bioequivalence Studies, U.S. Department of Health and Human Services Food and Drug Administration, Center for Drug Evaluation and Research (CDER), Center for Biologics Evaluation and Research (CBER) issued December 2002 and available at http://www.fda.gov/cder/guidance/index.htm. The exemplary high-fat meal contains approximately 50 percent of the total caloric content of the meal as fat and contains approximately 800 to 1000 calories; 500-600 calories from fat. As used herein, the term “fat” is used in its conventional, art-recognized meaning.

In one embodiment, use of quinine comprises administering quinine to a patient with a high fat meal, wherein the time to achieve Tₘₐₓ that is about 15 minutes to about 4 hours greater than Tₘₐₓ under fasted conditions, more specifically about 1 hour to about 2 hours greater than Tₘₐₓ under fasted conditions.

In another embodiment, a method of using quinine comprises informing a user a) that the oral administration of quinine under fed conditions does not substantially affect Cₘₐₓ or AUC of quinine when compared to fasted conditions; b) that the oral administration of about 324 or about 648 mg of quinine sulfate under fed conditions does not substantially affect Cₘₐₓ or AUC of quinine when compared to fasted conditions; c) that the oral administration of quinine under fed conditions increases Tₘₐₓ of quinine when compared to fasted conditions; or d) that the oral administration of about 324 or about 648 mg of quinine sulfate under fed conditions increases Tₘₐₓ of quinine when compared to fasted conditions.

As used herein, “oral administration of quinine under fed conditions does not substantially affect Cₘₐₓ or AUC of quinine when compared to fasted conditions” means that the comparison between Cₘₐₓ or AUC of a single administration of a single strength of quinine under fed conditions to a single administration of the same strength of quinine under fasted conditions results in a percent ratio of Cₘₐₓ or AUC having a 90% confidence interval upper limit of less than or equal to 125% or a lower limit of greater than or equal to 80%.

The method can further include the user timing the administration of quinine and a substance that is known to prolong the QT interval such that Tₘₐₓ of quinine differs from Tₘₐₓ of the substance by the longest time possible, specifically about one hour or greater, and more specifically about two hours or greater. By timing the administration of quinine and the substance, it may be possible to minimize any potential additive effects upon QTc prolongation. Therefore, if the user is informed of the food effect upon quinine Tₘₐₓ a dosing schedule can be developed for the patient with the intent to minimize any potential QTc prolonging additive effect.

It has recently been found through additional studies that the quinine sulfate pharmacokinetic profile is not linearly proportional when dosing an amount between 324 mg to 658 mg. Lack of dose proportionality (“non-linear pharmacokinetics”) of two dosage strengths of quinine sulfate (324 mg and 648 mg) was explored under fasted conditions. After a single administration of each dosage strength, the results for the geometric mean of the maximum plasma concentration (Cₘₐₓ) was found to be 4126.31 ng/ml when the Cₘₐₓ for a single 324 mg dose was multiplied by 2 to show the expected linear Cₘₐₓ for a double dose, (for purposes of comparison), while the Cₘₐₓ for the 648 mg dose was only 3174.89 ng/ml to result in a percent ratio of 129.97 with 90% confidence interval of 122.15 to 138.29). These results indicate that doubling the dose of quinine produces a Cₘₐₓ that is much lower than would be expected with linear dose proportionality under fasted conditions.

As used herein, “non-linear pharmacokinetics” means that the comparison between Cₘₐₓ or AUC of a single administration of a dose of a lesser strength of quinine (pharmacokinetic data adjusted proportionally to the corresponding strength of the greater dose) to a single administration of a dose of a greater strength of quinine results in a percent ratio of Cₘₐₓ or AUC having a 90% confidence interval upper limit of greater than 125% or a lower limit of less than 80%. For clarification, if the greater dose is 648 mg quinine sulfate and the lesser dose is 324 mg quinine sulfate, the Cₘₐₓ results of the administration of the lesser dose would be doubled for the comparison.

As it has been recently revealed that the maximum increase in QTc interval corresponds with peak quinine plasma concentration (Cₘₐₓ), knowledge of the lack of dose proportionality of quinine can be important when trying to avoid spikes or unsafe, elevated levels of quinine plasma concentration. With a dose proportional active agent, it is much easier to predict plasma levels between one dosage strength and another. With active agents that are not dose proportional, it is often not trivial to predict the pharmacokinetic outcome of altering dosage strengths, thereby making it uncertain what the efficacy and toxicity effects will be. Knowledge of the lack of dose proportionality may help a prescribing physician; for example, prescribe an optimized dosage for the patient while at the same time avoiding spikes or unnecessary increases in plasma concentrations of the active agent. Improved methods of using quinine or methods of increasing the safe administration of quinine can include administering quinine to a patient in need of quinine treatment and informing the user that dosing of quinine sulfate is not dose proportional between 324 mg and 648 mg.

Further studies reveal that quinine sulfate does exhibit dose proportionality at lower doses. For example, it has recently been found that the quinine sulfate pharmacokinetic profile is linearly proportional to dose amount between 280 mg to 324 mg. As used herein, “substantially linear pharmacokinetics” means that the comparison between Cₘₐₓ of a single administration of a dose of a lesser strength of quinine (pharmacokinetic data adjusted proportionally to the corresponding strength of the greater dose) to a single administration of a dose of a greater strength of quinine results in a percent ratio of Cₘₐₓ having a 90% confidence interval upper limit of less than or equal to 125% or a lower limit of greater than or equal to 80%.

In one embodiment, a method of using quinine comprises informing a user a) that the plasma levels of quinine sulfate when dosed orally under fasted conditions displays non-linear pharmacokinetics (AUC and Cₘₐₓ) when dosed between about 324 mg and about 648 mg; or b) that the plasma levels of the quinine sulfate when dosed orally under fasted conditions displays substantially linear pharmacokinetics (AUC and Cₘₐₓ) when dosed between about 260 mg to about 324 mg.

In yet another embodiment, a method of using quinine involves using quinine for the treatment of sp. Falciparum infection, uncomplicated Plasmodium falciparum malaria, severe or complicated Plasmodium falciparum malaria, treatment of babesiosis caused by Babesia microti, or the treatment or prevention of leg cramps, while additionally informing the user of the information provided herein regarding QT prolongation, food effect, dose proportionality, or the like.

Quinine can be formulated for administration where the formulation generally contains quinine and a pharmaceutically acceptable excipient. As used herein, “pharmaceutically acceptable excipient” means any other component added to the pharmaceutical formulation other than the active agent. Excipients may be added to facilitate manufacture, enhance stability, control release, enhance product characteristics, enhance bioavailability, enhance patient acceptability, etc. Pharmaceutical excipients include carriers, fillers, binders, disintegrants, lubricants, glidants, compression aids, colors, sweeteners, preservatives, suspending agents, dispersing agents, film formers, flavors, printing inks, buffer agents, pH adjusters, preservatives etc.

Also included herein are pharmaceutical products (kits) useful, for example, for the treatment or prevention of parasitic diseases caused by Plasmodium species (e.g. sp. Plasmodium, Plasmodium falciparum, etc.), the treatment and prophylaxis of leg cramps, or the treatment of babesiosis caused by Babesia microti, which comprise one or more containers containing a quinine formulation and optionally information or published material, e.g. as product inserts or product labels. The information can indicate quantities of the components to be administered, guidelines for administration, safety issues, and the like.

The kits may further comprise one or more conventional pharmaceutical kit components, such as, for example, one or more containers to aid in facilitating compliance with a particular dosage regimen; one or more carriers; etc. Exemplary kits can be in the form of bubble or blister pack cards, optionally arranged in a desired order for a particular dosing regimen. Suitable blister packs that can be arranged in a variety of configurations to accommodate a particular dosing regimen are well known in the art or easily ascertained by one of ordinary skill in the art.

Those forms existing as liquids, solutions, emulsions, or suspensions can be packaged for convenient dosing of pediatric or geriatric patients. For example, prefilled droppers (such as eye droppers or the like), prefilled syringes, and similar containers housing the liquid, solution, emulsion, or suspension form are contemplated.

In one embodiment, the quinine formulation is packaged with information informing the user that quinine may cause QT/QTc prolongation as an adverse reaction in some patients.

In one embodiment, a method of manufacturing a quinine pharmaceutical product comprises packaging a quinine dosage form with information a) that administration of quinine provides a mean±SD maximum QTc change from baseline around the quinine Tₘₐₓ of 10±19 msec for a 324 mg dose of quinine sulfate and 12±18 msec for a 648 mg dose of quinine sulfate; b) that a maximum QTc change from baseline of no greater than about 60 msec is observed in patients receiving a 324 mg dose of quinine sulfate or a 648 mg dose of quinine sulfate; c) that a QTc interval of no greater than about 500 msec is experienced in patients receiving a 324 mg dose of quinine sulfate or a 648 mg dose of quinine sulfate; or d) that the maximum increase in QTc interval corresponds with peak quinine plasma concentration (Cₘₐₓ).

Additional information can include that the patient is to use caution when taking more than a prescribed dose; to not double a next dose when a dose is missed; that the patient is not to take more doses than prescribed; that the patient is not to take more than 538 mg free base equivalent of quinine at one time; that the patient is not to take more than three doses in one day (538 mg free base equivalent quinine TID); that the patient is not to take more than 1614 mg free base equivalent of quinine (1944 mg quinine sulfate) in one day; that if the patient has missed a dose and more than about four hours has elapsed since the missed dose, it is recommended that the missed dose not be taken, but the patient is recommended take the next dose as previously scheduled; that caution is recommended if quinine is administered with a substance known to cause QT prolongation; that caution is recommended if quinine is administered to a patient known to have prolongation of QTc interval; that caution is recommended if quinine is administered to an elderly patient; that caution is recommended if quinine is administered to a patient with a clinical condition known to prolong the QT interval; that caution is recommended if quinine is administered with a substance that inhibits the metabolism of quinine; or that it is recommended that a patient be monitored for adverse reactions when the patient is administered quinine concomitantly with a substance that inhibits the metabolism of quinine.

In another embodiment, an article of manufacture comprises a container containing a dosage form of quinine, wherein the container is associated with published material informing a) that administration of quinine provides a mean±SD maximum QTc change from baseline around the quinine Tₘₐₓ of 10±19 msec for a 324 mg dose of quinine sulfate and 12±18 msec for a 648 mg dose of quinine sulfate; b) that a maximum QTc change from baseline of no greater than about 60 msec is observed in patients receiving a 324 mg dose of quinine sulfate or a 648 mg dose of quinine sulfate; c) that a QTc interval of no greater than about 500 msec is experienced in patients receiving a 324 mg dose of quinine sulfate or a 648 mg dose of quinine sulfate; or d) that the maximum increase in QTc interval corresponds with peak quinine plasma concentration (Cₘₐₓ).

In yet another embodiment, a method of manufacturing a quinine pharmaceutical product comprises packaging a quinine dosage form with information a) that the oral administration of quinine under fed conditions does not substantially affect Cₘₐₓ or AUC of quinine when compared to fasted conditions; b) that the oral administration of about 324 or about 648 mg of quinine sulfate under fed conditions does not substantially affect Cₘₐₓ or AUC of quinine when compared to fasted conditions; c) that the oral administration of quinine under fed conditions increases Tₘₐₓ of quinine when compared to fasted conditions; or d) that the oral administration of about 324 or about 648 mg of quinine sulfate under fed conditions increases Tₘₐₓ of quinine when compared to fasted conditions.

In another embodiment, an article of manufacture comprises a container containing a dosage form of quinine, wherein the container is associated with published material informing a) that the oral administration of quinine under fed conditions does not substantially affect Cₘₐₓ or AUC of quinine when compared to fasted conditions; b) that the oral administration of about 324 or about 648 mg of quinine sulfate under fed conditions does not substantially affect Cₘₐₓ or AUC of quinine when compared to fasted conditions; c) that the oral administration of quinine under fed conditions increases Tₘₐₓ of quinine when compared to fasted conditions; or d) that the oral administration of about 324 or about 648 mg of quinine sulfate under fed conditions increases Tₘₐₓ of quinine when compared to fasted conditions.

Additional information can include that the quinine can be taken with or without food; that it is recommended to take quinine with food to minimize gastric upset; that if quinine is administered with a substance known to prolong the QT interval, it is recommended that quinine be administered with or without food such that Tₘₐₓ of quinine differs from Tₘₐₓ of the substance by about one hour or greater, specifically by about two hours or greater.

In one embodiment, a method of manufacturing a quinine pharmaceutical product comprises packaging a quinine dosage form with information a) that the plasma levels of quinine sulfate when dosed orally under fasted conditions displays non-linear pharmacokinetics (AUC and Cₘₐₓ) when dosed between about 324 mg and about 648 mg; or b) that the plasma levels of the quinine sulfate when dosed orally under fasted conditions displays substantially linear pharmacokinetics (AUC and Cₘₐₓ) when dosed between about 260 mg to about 324 mg.

In one embodiment, an article of manufacture comprises a container containing a dosage form of quinine, wherein the container is associated with published material informing a) that the plasma levels of quinine sulfate when dosed orally under fasted conditions displays non-linear pharmacokinetics (AUC and Cₘₐₓ) when dosed between about 324 mg and about 648 mg; or b) that the plasma levels of the quinine sulfate when dosed orally under fasted conditions displays substantially linear pharmacokinetics (AUC and Cₘₐₓ) when dosed between about 260 mg to about 324 mg.

In one embodiment, the administration of a therapeutically effective amount of quinine or a quinine formulation to a patient causes the patient to experience a prolongation in the mean QT/QTc interval from baseline of less than about 20 ms, specifically less than about 10 ms, and more specifically less than about 5 ms.

In one embodiment, a quinine oral dosage form can comprise about 50 to about 1000 mg of quinine, more specifically about 100 to about 750 mg of quinine, and yet more specifically about 250 to about 500 mg of quinine base equivalent per dosage form.

In one embodiment, a quinine oral dosage form can comprise about 350 to about 520 mg of quinine, more specifically about 450 to about 500 mg of quinine, and yet more specifically about 475 to about 490 mg of quinine base equivalent per dosage form.

In another embodiment, a quinine oral dosage form can comprise about 100 to about 400 mg of quinine, more specifically about 150 to about 350 mg of quinine, and yet more specifically about 200 to about 300 mg of quinine base equivalent per dosage form.

In yet another embodiment, a quinine oral dosage form can comprise about 200 to about 600 mg of quinine sulfate, more specifically about 260 to about 520 mg of quinine sulfate, and yet more specifically about 300 to about 450 mg of quinine sulfate per dosage form.

The following examples further illustrate the invention but, of course, should not be construed as in any way limiting its scope.

### EXAMPLES

### Example 1

### QTc Interval Measurements Following Single Doses of Quinine Sulfate

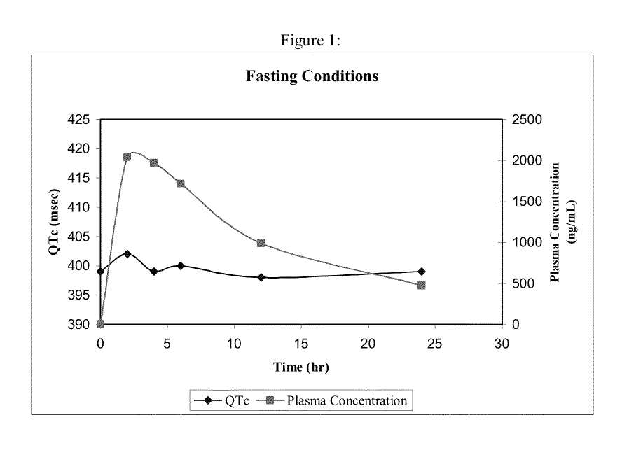

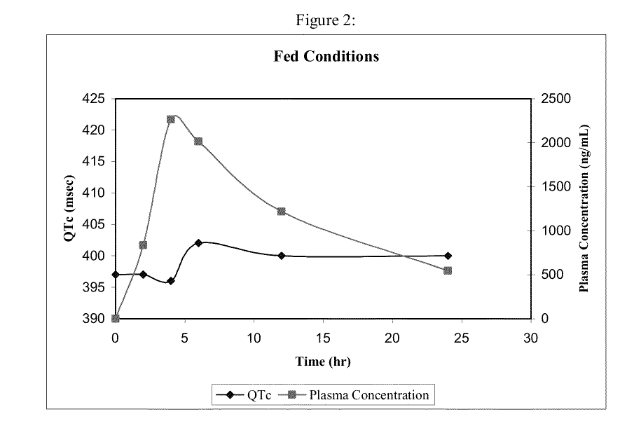

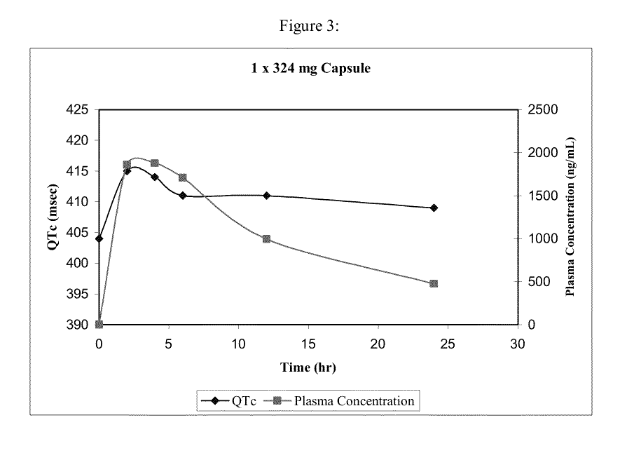

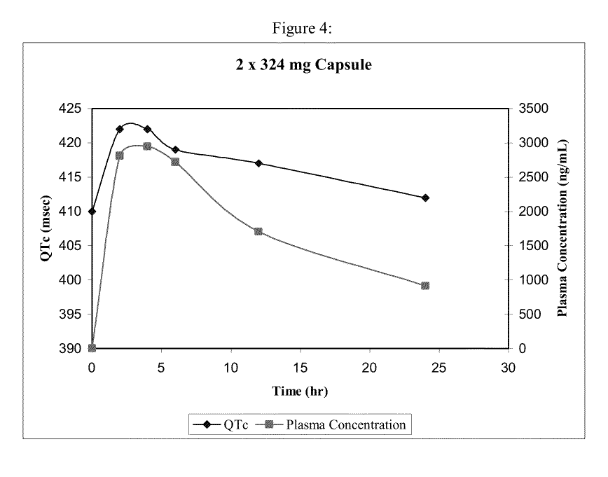

Studies were performed in healthy volunteers to measure QTc intervals following single doses of quinine sulfate. One study explored the effect of food on a single oral dose of a 324 mg oral capsule (324 mg quinine sulfate, 82 mg corn starch, 40 mg talc, 4 mg magnesium stearate). A second study was performed to compare two dose levels, a single oral dose of 324 mg quinine sulfate versus a single oral dose of 648 mg quinine sulfate (two capsules), both cases under fasting conditions. Repeated measurements of Electrocardiogram (ECG) intervals were taken for 50 subjects, 24 men and 26 women, who ranged in age from 18 to 47 years. The results are provided in Table 1 below and in FIGS. 1-4, which illustrate the correlation of mean maximum QTc interval prolongation effect to mean peak plasma quinine concentration.

TABLE 1

| | Study 1 A: 324 mg | | Study 1 B: 324 mg | | Study 2 C: 324 mg | | Study 2 D: 648 mg | |
|---|---|---|---|---|---|---|---|---|
| | Quinine Sulfate | | Quinine Sulfate | | Quinine Sulfate | | Quinine Sulfate | |
| | capsule* Fasting | | capsule* Fed | | capsule*, fasting | | capsules*, fasting | |
| | conditions | | conditions | | conditions | | conditions | |
| Time | Mean Plasma Concentration (ng/ml); QTc (msec) | | | | | | | |
| (hours) | (ng/ml) | (msec) | (ng/ml) | (msec) | (ng/ml) | (msec) | (ng/ml) | (msec) |
| 0 | 0 | 399 | 0 | 397 | 0; | 404 | 0 | 410 |
| 2 | 2040 | 402 | 835 | 397 | 1860 | 415 | 2808 | 422 |
| 4 | 1971 | 399 | 2265 | 396 | 1877 | 414 | 2946 | 422 |
| 6 | 1718 | 400 | 2013 | 402 | 1707 | 411 | 2721 | 419 |
| 12 | 990 | 398 | 1216 | 400 | 994 | 411 | 1705 | 417 |
| 24 | 473 | 399 | 543 | 400 | 475 | 409 | 912 | 412 |

*The capsules contained quinine sulfate USP, corn starch, magnesium stearate, and talc.

The data provided in columns A and B of Table 1 are directed to the mean plasma concentrations and QTc measurements over 24-hours following a single oral dose of a 324 mg Quinine Sulfate capsule under fasting (A) and fed (B) conditions. The data provided in column C of Table 1 are directed to the mean plasma concentrations and QTc measurements over 24-hours following a single oral dose of one 324 mg Quinine Sulfate capsule under fasting conditions. The data provided in column D of Table 1 are directed to the mean plasma concentrations and QTc measurements over 24-hours following a single oral dose of two 324 mg Quinine Sulfate capsule under fasting conditions.

As indicated by the data in the table, an increase in the mean QTc value was found to correspond with the peak quinine plasma concentration, which is reached in an average of 2.4 to 4.4 hours after oral administration in the fasted state and 4 to 6 hours when given with food was observed. Increases are higher when the same dose is given with food (which results in higher peak concentrations) and with a single dose of 648 mg as compared to 324 mg. In the study, seven subjects had significant prolongations in QTc interval (>450 msec). As illustrated, the higher the blood levels of quinine, the higher the incidence of QTc prolongation was observed.

### Example 2a

### Non-Linear Dose Proportionality Following Single Doses of Quinine Sulfate

A study was performed in healthy volunteers to measure the AUC (0-24 hours and 0-INF) and Cₘₐₓ following single oral doses of 1 and 2 capsules, each containing 324 mg quinine sulfate, in the fasted state. The study was performed on 24 subjects. After administration of the doses, blood samples were taken from the subjects every half hour for the first four hours and then every hour up to 48 hours. The results were calculated as Ln-transformed data, geometric mean, as well as the least squares mean, non-transformed data. The geometric means are based on least squares means of ln-transformed values. The results, provided in Table 2a below, indicate that there is nonlinear dose proportionality where doubling the dose produces a Cₘₐₓ that is lower than would be expected with linear dose proportionality under fasted conditions. Cₘₐₓ resulted from multiplying plasma concentration by 2 for the 1 capsule treatment is summarized in Table 2a, and is 129% of that from the 2 capsule treatment with the 90% confidence interval from 122-138%. AUCₜ and AUC_{inf} showed proportional increase when given two capsules.

TABLE 2a

| | | | | | |
|---|---|---|---|---|---|
| | 324 mg | | | | |
| | Quinine | | | | |
| | Sulfate, 1 | 648 mg | | 90% Confidence | |
| | capsule* (dose | Quinine | | Interval | P-values for |
| PK | adjusted to 2 × | Sulfate, 2 | | (Lower Limit, | Product |
| variable | 324 mg) | capsules* | % Ratio | Upper Limit) | Effects |

Ln-transformed data, Geometric Mean

| | | | | | |
|---|---|---|---|---|---|
| Cₘₐₓ | 4126.31 | 3174.89 | 129.97 | (122.15, 138.29) | <0.0001 |
| (ng/ml) | | | | | |
| AUC₀₋ₜ | 61186.53 | 54440.26 | 112.39 | (106.56, 118.54) | 0.0011 |
| (ng- | | | | | |
| hr/ml) | | | | | |
| AUC_{0-INF} | 66715.41 | 59166.93 | 112.76 | (105.69, 120.3) | 0.0044 |
| (ng- | | | | | |
| hr/ml) | | | | | |

Non-transformed data, least squares mean

| | | | | | |
|---|---|---|---|---|---|
| Cmax | 4247.02 | 3243.11 | 130.96 | (123.28, 138.63) | <0.0001 |
| (ng/ml) | | | | | |
| AUC₀₋ₜ | 64277.02 | 56394.65 | 113.98 | (108.03, 119.93) | 0.0006 |
| (ng- | | | | | |
| hr/ml) | | | | | |
| AUC_{0-INF} | 70886.14 | 61817.27 | 114.67 | (107.37, 121.97) | 0.0023 |
| (ng- | | | | | |
| hr/ml) | | | | | |
| Tmax | 2.78 | 2.80 | 99.25 | (84.8, 113.7) | 0.9298 |
| kₑₗᵢₘ | 0.0592 | 0.0572 | 103.48 | (94.67, 112.28) | 0.5045 |
| t_{1/2} | 12.76 | 12.80 | 99.67 | (85.69, 113.66) | 0.9683 |

*The capsules contained quinine sulfate USP, corn starch, magnesium stearate, and talc.

### Example 2b

### Dose Proportionality Following Single, Low Doses of Quinine Sulfate

A pediatric study was performed in healthy volunteers to measure the AUC (0-24 hours and 0-INF) and Cₘₐₓ following single oral doses of 260 mg quinine sulfate and 324 mg quinine sulfate (1.25 times the lower dose of 260 mg), in the fasted state. The study was performed on 22 subjects. After administration of the doses, blood samples were taken from the subjects every half hour for the first four hours, every hour up to 8 hours, and then at hours ten, twelve, sixteen, twenty-four, thirty-six, and forty-eight. The results were calculated as Ln-transformed data, geometric mean, as well as the least squares mean, non-transformed data. The geometric means are based on least squares means of Ln-transformed values. The results, provided in Table 2b below, indicate that there is linear dose proportionality when dosing quinine sulfate at the lower doses of 260 mg and 324 mg.

TABLE 2b

| | | | | | |
|---|---|---|---|---|---|
| | 260 mg | | | | |
| | Quinine | | | | |
| | Sulfate, 1 | 324 mg | | 90% Confidence | |
| | capsule* (dose | Quinine | | Interval | P-values for |
| PK | adjusted to | Sulfate, 1 | | (Lower Limit, | Product |
| variable | 1.25 × 324 mg) | capsule* | % Ratio | Upper Limit) | Effects |

Ln-transformed data, Geometric Mean

| | | | | | |
|---|---|---|---|---|---|
| Cₘₐₓ | 2251.55 | 2242.70 | 100.39 | (95.8, 105.21) | 0.8861 |
| (ng/ml) | | | | | |
| AUC₀₋ₜ | 30019.28 | 30318.55 | 99.01 | (93.83, 104.48) | 0.7535 |
| (ng- | | | | | |
| hr/ml) | | | | | |
| AUC_{0-INF} | 32072.92 | 32111.76 | 99.88 | (94.4, 105.67) | 0.9708 |
| (ng- | | | | | |
| hr/ml) | | | | | |

Non-transformed data, least squares mean

| | | | | | |
|---|---|---|---|---|---|
| Cmax | 2310.90 | 2275.46 | 101.56 | (95.93, 107.19) | 0.6384 |
| (ng/ml) | | | | | |
| AUC₀₋ₜ | 31285.26 | 31298.12 | 99.96 | (94.63, 105.28) | 0.9895 |
| (ng- | | | | | |
| hr/ml) | | | | | |
| AUC_{0-INF} | 33582.46 | 33280.89 | 100.91 | (95.36, 106.46) | 0.7811 |
| (ng- | | | | | |
| hr/ml) | | | | | |
| Tmax | 2.61 | 2.75 | 95.04 | (84.53, 105.55) | 0.4255 |
| kₑₗᵢₘ | 0.0615 | 0.0668 | 92.05 | (85, 99.04) | 0.0641 |
| t_{1/2} | 11.94 | 11.13 | 107.27 | (100.34, 114.2) | 0.0856 |

*The capsules contained quinine sulfate USP, corn starch, magnesium stearate, and talc.

### Example 3

### Food Effect Study of Quinine Sulfate

A study was performed in healthy volunteers to measure the pharmacokinetic parameters of quinine following single oral doses of a capsule containing 324 mg quinine sulfate (324 mg quinine sulfate, 82 mg corn starch, 40 mg talc, 4 mg magnesium stearate per capsule), both in the fed and fasted state. The study was performed on 24 subjects. After administration of a single 324 mg quinine sulfate capsule, blood samples were taken from the subjects every half hour for the first four hours and then every hour up to 48 hours. The results were calculated as Ln-transformed data, geometric mean, as well as the least squares mean, non-transformed data. The geometric means are based on least squares means of Ln-transformed values. The results, provided in Table 3a below, indicate that there is no food effect on Cₘₐₓ or AUC when dosing a 324 mg quinine sulfate capsule, but that the time to maximum plasma concentration is extended (90% confidence interval of 80-125% on Cₘₐₓ and AUC were met when fed vs. fasting).

TABLE 3a

| | | | | | |
|---|---|---|---|---|---|
| | 324 mg | 324 mg | | | |
| | Quinine | Quinine | | | 90% Confidence |
| | Sulfate*, | Sulfate*, | | | Interval |
| PK | Fed | Fasted | | Mean square | (Lower Limit, |
| variable | conditions | conditions | % Ratio | error | Upper Limit) |

Ln-transformed data, Geometric Mean

| | | | | | |
|---|---|---|---|---|---|
| Cₘₐₓ | 2440.04 | 2165.84 | 112.66 | 0.02397 | (104.67, 121.26) |
| (ng/ml) | | | | | |
| AUC₀₋ₜ | 32744.85 | 31228.60 | 104.86 | 0.01277 | (99.37, 110.64) |
| (ng- | | | | | |
| hr/ml) | | | | | |
| AUC_{0-INF} | 34861.77 | 34192.93 | 101.96 | 0.01638 | (95.94, 108.35) |
| (ng- | | | | | |
| hr/ml) | | | | | |

Non-transformed data, least squares mean

| | | | | | |
|---|---|---|---|---|---|
| Cₘₐₓ | 2541.30 | 2249.42 | 112.98 | 164143.05 | (104.42, 121.53) |
| (ng/ml) | | | | | |
| AUC₀₋ₜ | 34486.02 | 32346.80 | 106.61 | 18786477.76 | (100.25, 112.98) |
| (ng- | | | | | |
| hr/ml) | | | | | |
| AUC_{0-INF} | 37221.86 | 35775.15 | 104.04 | 34439960.62 | (96.25, 111.84) |
| (ng- | | | | | |
| hr/ml) | | | | | |
| Tₘₐₓ | 3.98 | 2.60 | 153.25 | 0.4812 | (140.55, 165.94) |
| kₑₗᵢₘ | 0.0662 | 0.0564 | 117.30 | 0.00012 | (107.95, 126.64) |
| t_{1/2} | 11.18 | 13.41 | 83.36 | 6.7071 | (74.19, 92.54) |

*The quinine was dosed in the form of capsules containing quinine sulfate USP, cornstarch, magnesium stearate, and talc.

The study was repeated using two 324 mg quinine sulfate capsules and 22 subjects. The results are provided below in Table 3b:

TABLE 3b

| | | | | |
|---|---|---|---|---|
| | 2 × 324 mg | 2 × 324 mg | | |
| | Quinine | Quinine | | 90% Confidence |
| | Sulfate*, | Sulfate*, | | Interval |
| PK | Fed | Fasted | | (Lower Limit, |
| variable | conditions | conditions | % Ratio | Upper Limit) |

Ln-transformed data, Geometric Mean

| | | | | |
|---|---|---|---|---|
| Cₘₐₓ | 3332.41 | 3197.25 | 104.23 | (99.13, 109.59) |
| (ng/ml) | | | | |
| AUC₀₋ₜ | 48942.43 | 47258.21 | 103.56 | (100.77, 106.44) |
| (ng- | | | | |
| hr/ml) | | | | |
| AUC_{0-INF} | 51699.75 | 50005.77 | 103.39 | (99.75, 107.16) |
| (ng- | | | | |
| hr/ml) | | | | |

Non-transformed data, least squares mean

| | | | | |
|---|---|---|---|---|
| Cₘₐₓ | 3424.30 | 3293.84 | 103.96 | (99.13, 108.8) |
| (ng/ml) | | | | |
| AUC₀₋ₜ | 51228.07 | 49484.64 | 103.52 | (100.65, 106.4) |
| (ng- | | | | |
| hr/ml) | | | | |
| AUC_{0-INF} | 54409.12 | 52643.14 | 103.35 | (99.3, 107.41) |
| (ng- | | | | |
| hr/ml) | | | | |
| Tₘₐₓ | 4.19 | 2.68 | 155.97 | (136.98, 174.96) |
| kₑₗᵢₘ | 0.0665 | 0.0642 | 103.56 | (96.98, 110.14) |
| t_{1/2} | 10.71 | 11.38 | 94.09 | (85, 103.17) |

*The quinine was dosed in the form of capsules containing quinine sulfate USP, cornstarch, magnesium stearate, and talc.

Again, the results, provided in Table 3b, indicate that there is no food effect on Cₘₐₓ or AUC when dosing two 324 mg quinine sulfate capsules, but that the time to maximum plasma concentration is extended. Such a result is unexpected as 648 mg of quinine sulfate is at about its limit of solubility in the gut and it is known that food increases the solubility of active agents in the gastrointestinal tract. By increasing the solubility of quinine sulfate with food, it would have been expected that the Cₘₐₓ ratio between the administration of 648 mg of quinine sulfate fed:fast would be greater that the Cₘₐₓ ratio between the administration of 324 mg of quinine sulfate fed:fast.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating a patient with quinine and an additional active known to cause QT prolongation, comprising:
providing a dosing regimen to a human to ensure a Tₘₐₓ of quinine differs by at least about 1 hour with a Tₘₐₓ of the additional active, wherein the dosing regimen comprises administering the additional active, and
administering two 324 mg quinine sulfate tablets or capsules orally to the human in a fasted state to provide a fasted state Tₘₐₓ, a fasted state AUC, and a fasted state Cₘₐₓ; or
administering two 324 mg quinine sulfate tablets or capsules orally to the human with a high fat meal to provide a nonfasted state Tₘₐₓ, a nonfasted state AUC, and a nonfasted state Cₘₐₓ;
wherein the nonfasted state Tₘₐₓ of quinine sulfate is greater than the fasted state Tₘₐₓ by about 15 minutes to about 4 hours, and
wherein nonfasted state AUC and nonfasted state Cₘₐₓ are substantially the same as the fasted state AUC and the fasted state Cₘₐₓ.

2. The method of claim 1, wherein the nonfasted state Tₘₐₓ of quinine sulfate is greater than the fasted state Tₘₐₓ by about 1 hour to about 2 hours.

3. The method of claim 1, wherein the nonfasted state Tₘₐₓ is about 3 hours or greater.

4. The method of claim 1, wherein the nonfasted state Tₘₐₓ is about 4 hours or greater.

5. The method of claim 1, wherein the nonfasted state Tₘₐₓ is about 5 hours or greater.

6. The method of claim 1, wherein the high-fat meal comprises about 50 percent of the total caloric content of the meal as fat and about 800 to 1000 calories.

7. The method of claim 1, wherein the quinine sulfate is administered as a capsule.

8. The method of claim 1,
wherein a percent ratio of nonfasted state Cₘₐₓ:fasted state Cₘₐₓ for the quinine tablet or capsule has a 90% confidence interval upper limit of less than or equal to 125% or a lower limit of greater than or equal to 80%, and
wherein a percent ratio of nonfasted state AUC:fasted state AUC for the quinine tablet or capsule has a 90% confidence interval upper limit of less than or equal to 125% or a lower limit of greater than or equal to 80%.

9. The method of claim 1, which provides reduced incidents of side effects or adverse events.
